(19) United States
(12) Patent Application Publication
(10) Pub. No.: US 2023/0162046 A1
(43) Pub. Date: May 25, 2023

(54) METHOD AND SYSTEM FOR ANOMALY DETECTION AND REPORT GENERATION

(71) Applicant: L&T TECHNOLOGY SERVICES LIMITED, Chennai (IN)

(72) Inventors: GINEESH SUKUMARAN, Mysore (IN); ASHOK AJAD, Gorakhpur (IN); SHUBHAM GUPTA, Meerut (IN); SWAROOP KUMAR M L, Mysuru (IN); AYUSHI BANSAL, Meerut (IN); KARISHMA JOSHI, Lucknow (IN)

(21) Appl. No.: 17/745,899

(22) PCT Filed: Dec. 31, 2020
(86) PCT No.: PCT/IB2020/062576

§ 371 Date: May 17, 2022

(30) Foreign Application Priority Data
Mar. 31, 2020 (IN) 202041014479

(51) Int. Cl. G06N 3/092; G06N 5/04

(52) U.S. Cl. CPC G06N 3/092; G06N 5/04

(57) ABSTRACT

Method and system for validating a report generated by a report generating model is provided. The method includes receiving an output that corresponds to an anomaly detected in an image from report generating model and includes one textual sentence. The method includes receiving an actual inference corresponding to anomaly and includes one textual sentence. The method further includes tokenizing output to generate output tokens and tokenizing actual inference to generate inference tokens. The method further includes classifying output tokens into predetermined categories. The method further includes classifying inference tokens into predetermined categories. The method further includes comparing output tokens with a corresponding inference tokens and assigning a match score to output tokens. The method further includes determining a combined score for the output based on the match score and validating the output based on the combined score.

### DESCRIPTION

### Technical Field

This disclosure relates generally to X-ray imaging, and more particularly to an artificial intelligence (AI) based system and method of abnormality detection and X-ray report generation.

### Background

Diagnostic images, for example, X-ray images, computerized tomography (CT) scans, Magnetic Resonance Imaging (MRI) images, Positron Emission Tomography (PET) images, etc. are effective radiological examinations for identifying various kind of pulmonary diseases and thoracic diseases. For example, a radiologist may examine an X-ray image to prepare a report. The prepared report may include impressions, findings, patient history, and additional tests that forms the basis of treatment.

Further, X-ray films may be digitalized which may ease the handling and communication of data associated with the X-ray films. Typically, conventional mechanisms for the X-ray imaging may be based on Convolutional Neural Network (CNN) models trained to detect abnormalities from X-ray radiographs. Such CNN models may store algorithm reports and benchmark model performance on large-scale public datasets (such as Chexpert, MIMIC-CXR and PADCHEST). However, such conventional mechanisms may have limitations of inappropriate highlighting of certain regions in X-ray images. Further, these conventional mechanisms may prove costly. Moreover, these conventional mechanisms involve complex algorithm structures.

As such, there is a need for system and method with an artificial neural network (ANN) based image classification model that is cost effective and sensitive for detection and classification of anomalies from digital diagnostic images like digital X-ray radiographs.

### SUMMARY

In accordance with an embodiment, a method of validating a report generated by a report generating model is disclosed. The method may include receiving an output from the report generating model. The output may correspond to an anomaly detected in an image. The output may comprise at least one textual sentence. The method may further include receiving an actual inference corresponding to the anomaly. The actual inference may comprise at least one textual sentence. The method may further include tokenizing the output to generate a plurality of output tokens and the actual inference to generate a plurality of inference tokens. The method may further include classifying the plurality of output tokens into one or more predetermined categories to generate one or more sets of output tokens corresponding to the one or more predetermined categories. Each of the one or more sets of output tokens may include at least one textual element. The method may further include classifying the plurality of inference tokens into the one or more predetermined categories to generate one or more sets of inference tokens corresponding to the one or more predetermined categories. Each of the one or more sets of inference tokens may include at least one textual element. The method may further include comparing each set of the one or more sets of output tokens with a corresponding set of the one or more sets of inference tokens, and assigning a match score to each set of the one or more sets of output tokens based on the comparison. The match score may be indicative of the degree of match between an associated set of output tokens and a corresponding set of inference tokens. The method may further include determining a combined score for the output based on the match score assigned to each set of the one or more sets of output tokens and validating the output based on the combined score.

In accordance with another embodiment, a system of validating a report generated by a report generating model is disclosed. The system may include a processor and a memory communicatively coupled to the processor. The memory may store processor-executable instructions, which, on execution, causes the processor to receive an output from the report generating model. The output may correspond to an anomaly detected in an image. The output may comprise at least one textual sentence. The processor-executable instructions may further cause the processor to receive an actual inference corresponding to the anomaly. The actual inference may comprise at least one textual sentence. The processor-executable instructions may further cause the processor to tokenize: the output to generate a plurality of output tokens, and the actual inference to generate a plurality of inference tokens. The processor-executable instructions may further cause the processor to classify the plurality of output tokens into one or more predetermined categories to generate one or more sets of output tokens corresponding to the one or more predetermined categories. Each of the one or more sets of output tokens may comprise at least one textual element. The processor-executable instructions further cause the processor to classify the plurality of inference tokens into the one or more predetermined categories to generate one or more sets of inference tokens corresponding to the one or more predetermined categories. Each of the one or more sets of inference tokens may include at least one textual element. The processor-executable instructions may further cause the processor to compare each set of the one or more sets of output tokens with a corresponding set of the one or more sets of inference tokens, and assign a match score to each set of the one or more sets of output tokens based on the comparison. The match score may be indicative of the degree of match between an associated set of output tokens and a corresponding set of inference tokens. The processor-executable instructions may further cause the processor to determine a combined score for the output based on the match score assigned to each set of the one or more sets of output tokens and validating the output based on the combined score.

In yet another embodiment, a non-transitory computer-readable storage medium is disclosed. The non-transitory computer-readable storage medium has computer-executable instructions stored thereon of validating a report generated by a report generating model. The computer-executable instructions may cause a computer comprising one or more processors to perform operations comprising receiving: an output from the report generating model. The output may correspond to an anomaly detected in an image. The output may comprise at least one textual sentence. The operations may further include receiving an actual inference corresponding to the anomaly. The actual inference may comprise at least one textual sentence. The operations may further include tokenizing: the output to generate a plurality of output tokens; and the actual inference to generate a plurality of inference tokens. The operations may further include classifying the plurality of output tokens into one or more predetermined categories to generate one or more sets of output tokens corresponding to the one or more predetermined categories. Each of the one or more sets of output tokens may comprise at least one textual element. The operations may further include classifying the plurality of inference tokens into the one or more predetermined categories to generate one or more sets of inference tokens corresponding to the one or more predetermined categories. Each of the one or more sets of inference tokens may comprise at least one textual element. The operations may further include comparing each set of the one or more sets of output tokens with a corresponding set of the one or more sets of inference tokens. The operations may further include assigning a match score to each set of the one or more sets of output tokens based on the comparison. The match score may be indicative of the degree of match between an associated set of output tokens and a corresponding set of inference tokens. The operations may further include determining a combined score for the output based on the match score assigned to each set of the one or more sets of output tokens and validating the output based on the combined score.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a block diagram that illustrates an environment of a system for abnormality detection and report generation from a digital X-ray radiograph, in accordance with an embodiment of the disclosure.

FIG. 2 is a block diagram of an exemplary system for validating a report generated by a report generating model using an AI based model, in accordance with an embodiment of the disclosure.

FIG. 3 is a functional block diagram that illustrates various modules of a system for validating a report generated by a report generating model, in accordance with an embodiment of the disclosure.

FIG. 4 is a process flow diagram that illustrates exemplary operations for extracting radiological findings from a report generated by a report generating model using an AI based model, in accordance with an embodiment of the present disclosure.

FIG. 5 is a process flow diagram that illustrates exemplary operations for convolution attention-based sentence reconstruction and scoring (CARES) using an AI based model, in accordance with an embodiment of the present disclosure.

FIG. 6 is a process flow diagram that shows an exemplary scenario for scoring operation in a system for validating a report generated by a report generating model, in accordance with an embodiment of the present disclosure.

FIG. 7 is another process flow diagram that shows the exemplary scenario for scoring operation in a system for validating a report generated by a report generating model, in accordance with an embodiment of the present disclosure.

FIG. 8 is a flowchart that illustrates an exemplary method of validating a report generated by a report generating model, in accordance with an embodiment of the disclosure.

### DETAILED DESCRIPTION OF THE DRAWINGS

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims. Additional illustrative embodiments are listed below.

The following described implementations may be found in the disclosed method and system for validating a report generated by a report generating model, based on an Artificial Intelligence (AI) based model. Exemplary aspects of the disclosure provide a system for validating the report associated with detection and classification of anomalies from digital X-ray radiographs for real-time inference, while maintaining a prediction accuracy for image class associated with classification of anomalies from digital X-ray radiographs.

The disclosed system and method may be used for validating a report generated by a report generating model. The disclosed system may use AI models to detect clinically meaningful radiological findings (such as, chest X-ray findings) as effectively as experienced radiologists. The disclosed system may facilitate generation of reports based on the validation of the reports, and consequently, the results may be returned to patients quickly, which aids in medical decision making. The various biases, lack of knowledge, or clerical errors made in the process of observing diagnostic images (such as, digital X-ray radiographs) may be minimized with the disclosed system. The reports may be populated based on the validation of the reports that can save time for human clinicians, and identify measurements or values that qualify as abnormal, thereby reducing workflow burdens on the human clinicians.

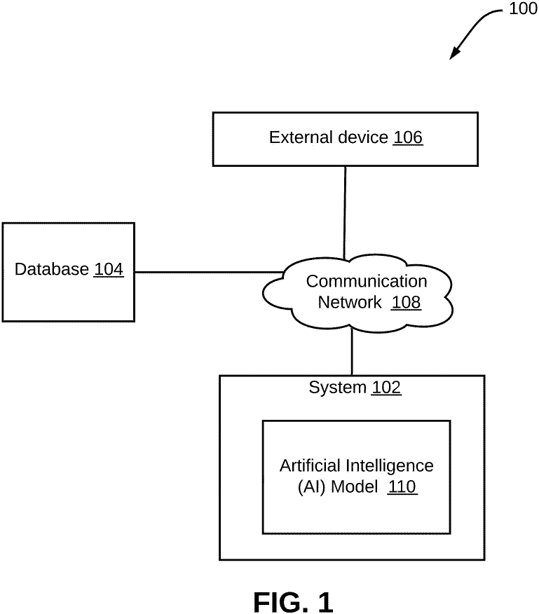

FIG. 1 is a block diagram that illustrates an environment for a system of validating a report generated by a report generating model, in accordance with an embodiment of the disclosure. With reference to FIG. 1, there is shown an environment 100. The environment 100 includes a system 102, a database 104, an external device 106, and a communication network 108. The system 102 may be communicatively coupled to the database 104 and the external device 106, via the communication network 108. In some embodiments, the system 102 may include an AI model 110, for example, as part of an application stored in memory of the system 102.

The system 102 may include suitable logic, circuitry, interfaces, and/or code that may be configured to validate the report received from the database 104 using the AI model 110. The report may be generated by a report generating model and stored in the database 104. The AI model 110 may be trained to extract a radiological finding from the report. In accordance with an embodiment, the report may include an image (such as, digital X-ray radiograph). To extract the radiological finding from the report, the AI model 110 may be configured to extract one or more features of anomalies from the image. Additionally, the AI model 110, may be trained to generate textual sentence(s) that correspond to a textual description that may be readable by a radiologist to know a condition of a patient in a structured manner. The AI model 110, once trained, may be deployable for applications (such as, a diagnostic application) which may take actions or generate real-time or near real-time inferences. By way of an example, the system 102 may be implemented as a plurality of distributed cloud-based resources by use of several technologies that are well known to those skilled in the art. Other examples of implementation of the system 102 may include, but are not limited to, medical diagnostic equipment, a web/cloud server, an application server, a media server, and a Consumer Electronic (CE) device.

The database 104 may include suitable logic, circuitry, interfaces, and/or code that may be configured to store data received, utilized and processed by the system 102. The data may correspond to output data from the report associated with anomaly detection in the image. The output data may include at least one textual sentence and an actual inference corresponding to anomaly. The actual inference may include at least one textual sentence. In accordance with an embodiment, the database 104 may store a plurality of images (such as, digital X-ray radiographs) that are used to train the AI model 110 by the system 102, or as an input to the trained AI model 110 of the system in a test environment (e.g., for benchmarking) or in an application-specific deployment, e.g., applications related to anomaly detection.

In accordance with an embodiment, the database 104 may store Digital Imaging and Communications in Medicine (DICOM) data. In accordance with an embodiment, the database 104 may store, exchange, and transmit medical images. The medical images may correspond to, but not limited to, radiography images, ultrasonography images, Computed Tomography (CT) scan images, Magnetic Resonance Imaging (MRI) images, and radiation therapy images. In accordance with another embodiment, the database 104 may store a final result and/or report generated by the system 102.

Although in FIG. 1, the system 102 and the database 104 are shown as two separate entities, this disclosure is not so limited. Accordingly, in some embodiments, the entire functionality of the database 104 may be included in the system 102, without a deviation from scope of the disclosure.

The external device 106 may include suitable logic, circuitry, interfaces, and/or code that may be configured to deploy the AI model 110, as part of an application engine that may use the output of the AI model 110 to generate real or near-real time inferences, take decisions, or output prediction results for diagnosis of diseases. The AI model 110 may be deployed on the external device 106 once the AI model 110 is trained on the system 102. The functionalities of the external device 106 may be implemented in portable devices, such as a high-speed computing device, and/or non-portable devices, such as a server. Examples of the external device 106 may include, but are not limited to, medical diagnosis equipment, a smart phone, a mobile device, or a laptop.

The communication network 108 may include a communication medium through which the system 102, the database 104, and the external device 106 may communicate with each other. Examples of the communication network 108 may include, but are not limited to, the Internet, a cloud network, a Wireless Fidelity (Wi-Fi) network, a Personal Area Network (PAN), a Local Area Network (LAN), or a Metropolitan Area Network (MAN). Various devices in the environment 100 may be configured to connect to the communication network 108, in accordance with various wired and wireless communication protocols. Examples of such wired and wireless communication protocols may include, but are not limited to, a Transmission Control Protocol and Internet Protocol (TCP/IP), User Datagram Protocol (UDP), Hypertext Transfer Protocol (HTTP), File Transfer Protocol (FTP), Zig Bee, EDGE, IEEE 802.11, light fidelity(Li-Fi), 802.16, IEEE 802.11s, IEEE 802.11g, multi-hop communication, wireless access point (AP), device to device communication, cellular communication protocols, and Bluetooth (BT) communication protocols.

The AI model 110 may be referred to as a computational network or a system of artificial neurons, where each Neural Network (NN) layer of the AI model 110 includes artificial neurons as nodes. Outputs of all the nodes in the AI model 110 may be coupled to at least one node of preceding or succeeding NN layer(s) of the AI model 110. Similarly, inputs of all the nodes in the AI model 110 may be coupled to at least one node of preceding or succeeding NN layer(s) of the AI model 110. Node(s) in a final layer of the AI model 110 may receive inputs from at least one previous layer. A number of NN layers and a number of nodes in each NN layer may be determined from hyperparameters of the AI model 110. Such hyperparameters may be set before or while training the AI model 110 on a training dataset of images.

Each node in the AI model 110 may correspond to a mathematical function with a set of parameters, tunable while the AI model 110 is trained. These parameters may include, for example, a weight parameter, a regularization parameter, and the like. Each node may use the mathematical function to compute an output based on one or more inputs from nodes in other layer(s) (e.g., previous layer(s)) of the AI model 110.

The AI model 110 may include electronic data, such as, for example, a software program, code of the software program, libraries, applications, scripts, or other logic/instructions for execution by a processing device, such as the system 102 and the external device 106. Additionally, or alternatively, the AI model 110 may be implemented using hardware, such as a processor, a microprocessor (e.g., to perform or control performance of one or more operations), a field-programmable gate array (FPGA), or an application-specific integrated circuit (ASIC). In some embodiments, the AI model 110 may be implemented using a combination of both the hardware and the software program.

The AI model 110 may include a first AI model and a second AI model (not shown in FIG. 1). In accordance with an embodiment, the first AI model may correspond to a deep machine learning model (such as, a convolutional neural network model). In accordance with an embodiment, the second AI model may correspond to, a natural language processing (NLP) model (such as, a Long Short-Term Memory (LSTM) model and Transformers). In accordance with an embodiment, The AI model 110 may implement a deep machine learning model and a natural language processing (NLP) model. In accordance with an embodiment, the training of the AI model 110 may be performed to detect a plurality of abnormalities from a set of radiography images (i.e. a chest X-ray images). The AI model 110 may be trained to classify each of the plurality of abnormalities into a set of abnormality on the basis of their type and create a plurality of visual indicators on each of the set of abnormality.

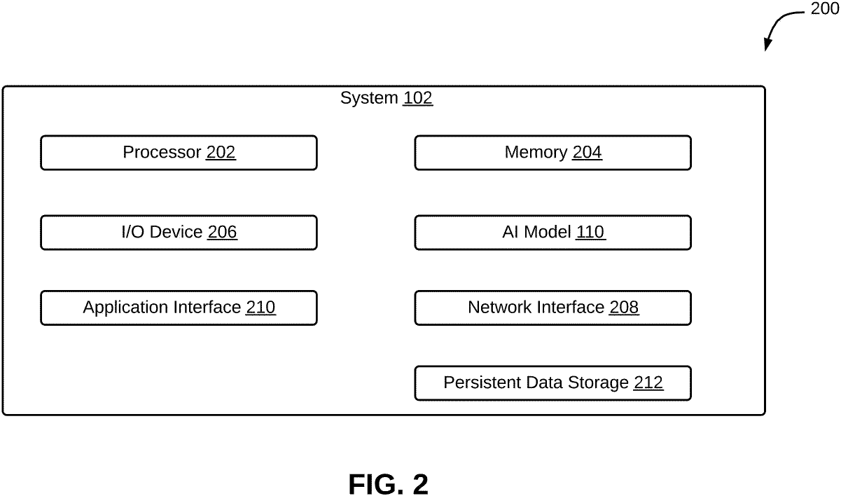

FIG. 2 is a block diagram of an exemplary system for validating a report generated by a report generating model using an AI based model, in accordance with an embodiment of the disclosure. FIG. 2 is explained in conjunction with elements from FIG. 1.

With reference to FIG. 2, there is shown a block diagram 200 of the system 102. The system 102 may include a processor 202, a memory 204, an input/output (I/O) device 206, a network interface 208, an application interface 210, and a persistent data storage 212. The system 102 may also include the AI model 110, as part of, for example, a software application for validating a report generated by a report generating model. The processor 202 may be communicatively coupled to the memory 204, the I/O device 206, the network interface 208, the application interface 210, and the persistent data storage 212. In one or more embodiments, the system 102 may also include a provision/functionality to store images.

The processor 202 may include suitable logic, circuitry, interfaces, and/or code that may be configured to validate output (or output data) from a report generating model. The processor 202 may be implemented based on a number of processor technologies, which may be known to one ordinarily skilled in the art. Examples of implementations of the processor 202 may be a Graphics Processing Unit (GPU), a Reduced Instruction Set Computing (RISC) processor, an Application-Specific Integrated Circuit (ASIC) processor, a Complex Instruction Set Computing (CISC) processor, a microcontroller, Artificial Intelligence (AI) accelerator chips, a co-processor, a central processing unit (CPU), and/or a combination thereof.

The memory 204 may include suitable logic, circuitry, and/or interfaces that may be configured to store instructions executable by the processor 202. Additionally, the memory 204 may be configured to store program code of the AI model 110 and/or the software application that may incorporate the program code of the AI model 110. Examples of implementation of the memory 204 may include, but are not limited to, Random Access Memory (RAM), Read Only Memory (ROM), Electrically Erasable Programmable Read-Only Memory (EEPROM), Hard Disk Drive (HDD), a Solid-State Drive (SSD), a CPU cache, and/or a Secure Digital (SD) card.

The I/O device 206 may include suitable logic, circuitry, and/or interfaces that may be configured to act as an I/O interface between a user and the system 102. The user may include a general practitioner or a radiologist who operates the system 102 for performing a screening test of a patient, or a patient who undergoes a screening test for anomaly detection. The I/O device 206 may include various input and output devices, which may be configured to communicate with different operational components of the system 102. Examples of the I/O device 206 may include, but are not limited to, a touch screen, a keyboard, a mouse, a joystick, a microphone, and a display screen.

The network interface 208 may include suitable logic, circuitry, interfaces, and/or code that may be configured to facilitate different components of the system 102 to communicate with other devices, such as the external device 106, in the environment 100, via the communication network 108. The network interface 208 may be configured to implement known technologies to support wired or wireless communication. Components of the network interface 208 may include, but are not limited to an antenna, a radio frequency (RF) transceiver, one or more amplifiers, a tuner, one or more oscillators, a digital signal processor, a coder-decoder (CODEC) chipset, an identity module, and/or a local buffer.

The network interface 208 may be configured to communicate via offline and online wireless communication with networks, such as the Internet, an Intranet, and/or a wireless network, such as a cellular telephone network, a wireless local area network (WLAN), personal area network, and/or a metropolitan area network (MAN). The wireless communication may use any of a plurality of communication standards, protocols and technologies, such as Global System for Mobile Communications (GSM), Enhanced Data GSM Environment (EDGE), wideband code division multiple access (W-CDMA), code division multiple access (CDMA), LTE, time division multiple access (TDMA), Bluetooth, Wireless Fidelity (Wi-Fi) (such as IEEE 802.11, IEEE 802.11b, IEEE 802.11g, IEEE 802.11n, and/or any other IEEE 802.11 protocol), voice over Internet Protocol (VoIP), Wi-MAX, Internet-of-Things (IoT) technology, Machine-Type-Communication (MTC) technology, a protocol for email, instant messaging, and/or Short Message Service (SMS).

The application interface 210 may be configured as a medium for the user to interact with the system 102. The application interface 210 may be configured to have a dynamic interface that may change in accordance with preferences set by the user and configuration of the system 102. In some embodiments, the application interface 210 may correspond to a user interface of one or more applications installed on the system 102.

The persistent data storage 212 may include suitable logic, circuitry, and/or interfaces that may be configured to store program instructions executable by the processor 202, operating systems, and/or application-specific information, such as logs and application-specific databases. The persistent data storage 212 may include a computer-readable storage media for carrying or having computer-executable instructions or data structures stored thereon. Such computer-readable storage media may include any available media that may be accessed by a general-purpose or special-purpose computer, such as the processor 202.

By way of example, and not limitation, such computer-readable storage media may include tangible or non-transitory computer-readable storage media including, but not limited to, Compact Disc Read-Only Memory (CD-ROM) or other optical disk storage, magnetic disk storage or other magnetic storage devices (e.g., Hard-Disk Drive (HDD)), flash memory devices (e.g., Solid State Drive (SSD), Secure Digital (SD) card, other solid state memory devices), or any other storage medium which may be used to carry or store particular program code in the form of computer-executable instructions or data structures and which may be accessed by a general-purpose or special-purpose computer. Combinations of the above may also be included within the scope of computer-readable storage media.

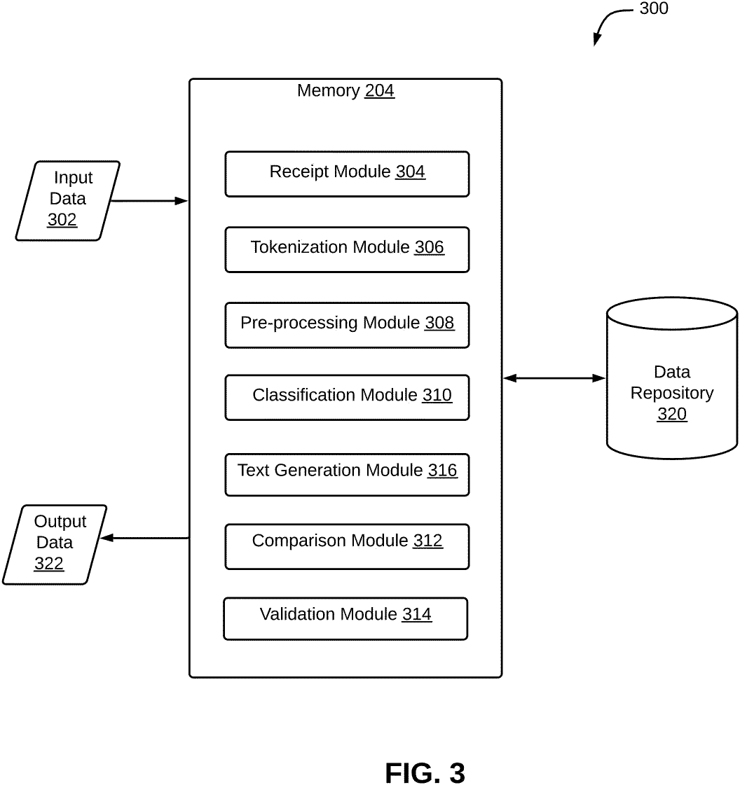

Computer-executable instructions may include, for example, instructions and data configured to cause the processor 202 to perform a certain operation or a set of operations associated with the system 102. The functions or operations executed by the system 102, as described in FIG. 1, may be performed by the processor 202. In accordance with an embodiment, additionally, or alternatively, the operations of the processor 202 are performed by various modules that are described in detail, for example, in FIG. 3.

FIG. 3 is a functional block diagram 300 that illustrates various modules of a system for validating a report generated by a report generating model, in accordance with an embodiment of the disclosure. FIG. 3 is explained in conjunction with elements from FIG. 1 and FIG. 2.

With reference to FIG. 3, there is shown input data 302, a receipt module 304, a tokenization module 306, a preprocessing module 308, a classification module 310, a comparison module 312, a validation module 314, a text generation module 316, a data repository 318, and output data 320.

The receipt module 304 of the system 102 may be configured to receive the input data 302. For example, the input data 302 may be received from the database 104. In accordance with an embodiment, the input data 302 may correspond to one or more images associated with a subject. In accordance with an embodiment, the one or more images may correspond to, but not limited to, X-ray radiographs, Computed Tomography (CT) scans, and Magnetic Resonance Imaging (MRI) scans. In accordance with an embodiment, the receipt module 304 may be configured to process the input data 302 based on image reconstruction techniques. The examples for the image reconstruction techniques may include, but not limited to, a back-projection technique, an inverse Fourier transform technique, and a sparse reconstruction technique.

The input data 302 to the system 102 may correspond to, for example, a diagnostic scan or a textual report of a user (patient). The input data 302 may correspond to an anomaly detected in an image. The output may comprise at least one textual sentence. In accordance with an embodiment, the classification module 310 may be configured to extract one or more features indicative of one or more anomalies from the image using a first AI model. It may be noted that the classification module 310 may implement a first AI model. It may be further noted that the first AI model may correspond to a Convolutional Neural Network (CNN) model. The CNN model may be modelled on a deep neural network architecture with multiple stages. The classification module 310 may be further configured to assign a label to each of the one or more features. In accordance with an embodiment, the classification module 310 may be configured to classify the image upon assigning a label (or class label) to the image. In accordance with an embodiment, the classification module 310 may be configured to localize anomalies to identify location in the image.

In accordance with an embodiment, the text generation module 316 may be configured to generate at least one textual sentence based on the label assigned to each of the one or more features using a second AI model. In accordance with an embodiment, the second AI model may correspond to a Long Short-Term Memory (LSTM) model and/or a transformer model. In accordance with an embodiment, the receipt module 304 of the system 102 may be further configured to receive an actual inference corresponding to the anomaly. The actual inference may comprise at least one textual sentence. It may be noted that the actual inference, in some embodiments, may be provided by a medical practitioner/doctor based upon studying the one or more images associated with a subject (i.e., X-ray radiographs, CT scans, MRI scans, etc.).

After receiving of the image (input) data 302 (such as, the output from the report generating model) and the actual inference corresponding to the anomaly, the tokenization module 306 may be configured to tokenize the output to generate a plurality of output tokens. For tokenization of the output, the at least one sentence may be split into a phrase or smaller units (output tokens), such as individual words or terms. In accordance with an embodiment, the tokenization module 306 may be further configured to tokenize the actual inference to generate a plurality of inference tokens.

In accordance with an embodiment, the preprocessing module 308 may be configured to preprocess the plurality of output tokens to extract relevant tokens from each of the one or more sets of output tokens. Further, in accordance with an embodiment, the preprocessing module 308 may be configured to preprocess the plurality of inference tokens to extract relevant tokens from each of the one or more sets of inference tokens. In accordance with an embodiment, the preprocessing of a set of output tokens or a set of inference tokens associated with a category may be performed by the preprocessing module 308 based on a historical database corresponding to the category. The historical database may correspond to the data repository 318.

In accordance with an embodiment, the classification module 310 may be further configured to classify the plurality of output tokens into one or more predetermined categories. Based on the classification, the classification module 310 may be configured to generate one or more sets of output tokens corresponding to the one or more predetermined categories. In other words, the plurality of output tokens may be classified into the one or more predetermined categories to obtain one or more sets corresponding to each of the categories. In accordance with an embodiment, each of the one or more sets of output tokens may comprise at least one textual element. For example, one category may be an anomaly location category, i.e., a location in the body part (e.g., chest) captured in the image where the anomaly may be present. Another category may be an anomaly type category, i.e., a type of anomaly (e.g., congestion, etc.) present in the body part captured in the image. A yet another category may be an anomaly extent category, i.e., an extent of anomaly (e.g., subtle/excess, large/small, etc.) present in the body part captured in the image.

In accordance with an embodiment, the classification module 310 may be configured to classify the plurality of inference tokens into the one or more predetermined categories. Based on the classification, the classification module 310 may be configured to generate one or more sets of inference tokens corresponding to the one or more predetermined categories. In accordance with an embodiment, each of the one or more sets of inference tokens comprises at least one textual element.

In accordance with an embodiment, the comparison module 312 may be configured to compare the relevant tokens from each of the one or more sets of output tokens with the relevant tokens from a corresponding set of the one or more sets of inference tokens. Further, in accordance with an embodiment, the comparison module 312 may be configured to compare each set of the one or more sets of output tokens with a corresponding set of the one or more sets of inference tokens.

In accordance with an embodiment, the validation module 314 may be configured to assign a match score to each set of the one or more sets of output tokens based on the comparison. The match score may be indicative of the degree of match between an associated set of output tokens and a corresponding set of inference tokens. In accordance with an embodiment, the validation module 314 may be further configured to determine a combined score for the output based on the match score assigned to each set of the one or more sets of output tokens. In accordance with an embodiment, the validation module 314 may be configured to validate the output based on the combined score. The validated output may correspond to the output data 320. In accordance with an embodiment, the output data 320 may be rendered as a report (a validated report) on a user device.

The data repository 318 may be configured to receive predicted image class outputs with class labels and probabilities from the classification module 310 and the text generation module 316. Further, the data repository 318 may be configured to store information that is required for processing in run time. Such information may include X-ray radiographs, predicted classes, dataset of training images, dataset of test images, and reports generated by the report generating model. The data repository 318 may correspond to a high-speed data repository, such as, but not limited to, Redis, and NoSQL.

It should be noted that the system 102 may be implemented in programmable hardware devices such as programmable gate arrays, programmable array logic, programmable logic devices, or the like. Alternatively, the system 102 may be implemented in software for execution by various types of processors. An identified engine/module of executable code may, for instance, include one or more physical or logical blocks of computer instructions which may, for instance, be organized as a component, module, procedure, function, or other construct. Nevertheless, the executables of an identified engine/module need not be physically located together but may include disparate instructions stored in different locations which, when joined logically together, comprise the identified engine/module and achieve the stated purpose of the identified engine/module. Indeed, an engine or a module of executable code may be a single instruction, or many instructions, and may even be distributed over several different code segments, among different applications, and across several memory devices.

As will be appreciated by one skilled in the art, a variety of processes may be employed for validating a report generated by a report generating model. For example, the exemplary system 102 may validate the report, by the process discussed herein. In particular, as will be appreciated by those of ordinary skill in the art, control logic and/or automated routines for performing the techniques and steps described herein may be implemented by the system 102 either by hardware, software, or combinations of hardware and software. For example, suitable code may be accessed and executed by the one or more processors on the system 102 to perform some or all of the techniques described herein. Similarly, application specific integrated circuits (ASICs) configured to perform some or all the processes described herein may be included in the one or more processors on the system 102.

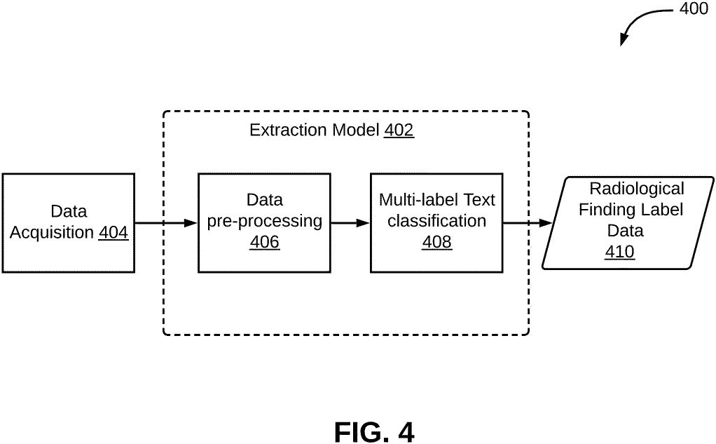

FIG. 4 is a process flow diagram 400 that illustrates exemplary operations for extracting radiological findings from a report generated by a report generating model using an AI based model, in accordance with an embodiment of the present disclosure. FIG. 4 is explained in conjunction with elements from FIG. 1, FIG. 2, and FIG. 3. With reference to FIG. 4, there is shown a diagram 400 that illustrates a set of operations for using an extraction model 402. In an embodiment, the extraction model 402 may be a Bidirectional Encoder Representations from Transformers (BERT) model. In another embodiment, the extraction model 402 may be any Natural Language Processing (NLP)-based encoder model.

The extraction model 402 may correspond to the AI model 110 of FIG. 1 and may be, for example, modelled on a deep neural network architecture with multiple stages. The extraction model may use attention mechanism for label extraction. The extraction model 402 may be pre-trained and fine-tuned for classification task of detecting anomalies in diagnostic images. In accordance with an embodiment, the last layer of the extraction model 402 may be trained for classification task.

A data acquisition 404 operation may be performed. In accordance with an embodiment, the system 102 may be configured to acquire input data. The input data may be received from the database 104. In accordance with an embodiment, the input data may correspond to one or more images associated with a subject. In accordance with an embodiment, the one or more images may correspond to, but not limited to, X-ray radiographs, CT scans, PET scan, microscopy images and MRI scans. The input data may correspond to an output from a report generating model. The input data may be indicative of an anomaly detected in an image. The output from the report generating model may comprise at least one textual sentence.

A data pre-processing operation 406 may be performed. In accordance with an embodiment, the system 102 may be configured to pre-process the input data, such as, the at least one textual sentence from the report generating model. into a format that the extraction model 402 understands. The data pre-processing operation on the at least one textual sentence may include, but not limited to, a sentence extraction, a sentence clean up and a spell correction technique. In some embodiments, after sentence formatting by using the data processing operations, the processed input data may be fed to an encoder (not shown in FIG. 4) of the extraction model 402.

The extraction model 402 may be trained on a training set of images that can be assigned with multiple categories represented as a set of target labels, and the extraction model 402 may predict the label set of test data (input data). The processed input data may be tokenized by the system using the extraction model 402. The tokenization may include breaking up of input data (at least one textual sentence) into individual words (tokens). The tokens used by the extraction model 402 may identify start and end of the at least one sentence, may append “index” and “segment” tokens to each input to the extraction model 402.

A multi-label text classification operation 408 may be performed. In accordance with an embodiment, in the multi-label text classification, the pre-processed input data may be classified in to one or more than one class. Multi labels may be non-exclusive labels. The system 102 may be configured to classify the text (the at least one sentence) with label and may be classified in to one or more than one class. The classified input data may correspond to radiological findings label data 410.

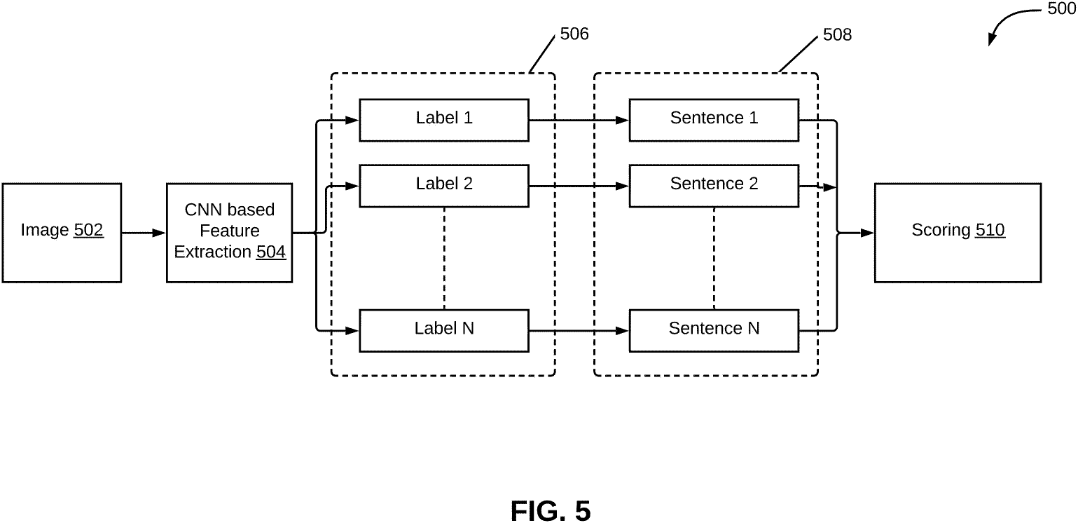

FIG. 5 is a process flow block diagram 500 that illustrates exemplary operations for convolution attention-based sentence reconstruction and scoring (CARES) using an AI based model, in accordance with an embodiment of the present disclosure. FIG. 5 is explained in conjunction with elements from FIG. 1 to FIG. 4.

At step 502, an image may be received. At step 504, CNN based feature extraction may be performed on the received image. The the classification module 310 may be configured to extract one or more features indicative of one or more anomalies from the image using a first AI model. In accordance with an embodiment, the first AI model may correspond to a Convolutional Neural Network (CNN) model.

At step 506, label assigning operation may be performed. To this end, the classification module 310 may be configured to assign labels (such as, label 1, label 2,..., label N) to the one or more features. The CNN model may use attention-based mechanism to assign the labels.

At step 508, sentence generation operation may be performed. The text generation module 316 may be configured to generate at least one textual sentence (such as, sentence 1, sentence 2, up to sentence N) based on the labels (such as, label 1, label 2...label N) assigned to each of the one or more features using a second AI model. The second AI model may correspond to a Long Short-Term Memory (LSTM) model and a transformer model.

At step 510, scoring operation may be performed. The sentences (such as, sentence 1, sentence 2, up to sentence N) may be tokenized into smaller units (output tokens). In accordance with an embodiment, the tokenization module 306 may be configured to tokenize actual inference to generate a plurality of inference tokens. In accordance with an embodiment, the classification module 310 may be configured to classify output tokens into one or more predetermined categories. Based on the classification, the classification module 310 may be configured to generate one or more sets of output tokens corresponding to the one or more predetermined categories. Each of the one or more sets of output tokens may comprise at least one textual element.

The classification module 310 may be further configured to classify inference tokens into the one or more predetermined categories. Based on the classification, the classification module 310 may be configured to generate one or more sets of inference tokens corresponding to the one or more predetermined categories. Each of the one or more sets of inference tokens comprises at least one textual element. The comparison module 312 may be configured to compare the relevant tokens from each of the one or more sets of output tokens with the relevant tokens from a corresponding set of the one or more sets of inference tokens. Further, the comparison module 312 may be configured to compare each set of the one or more sets of output tokens with a corresponding set of the one or more sets of inference tokens.

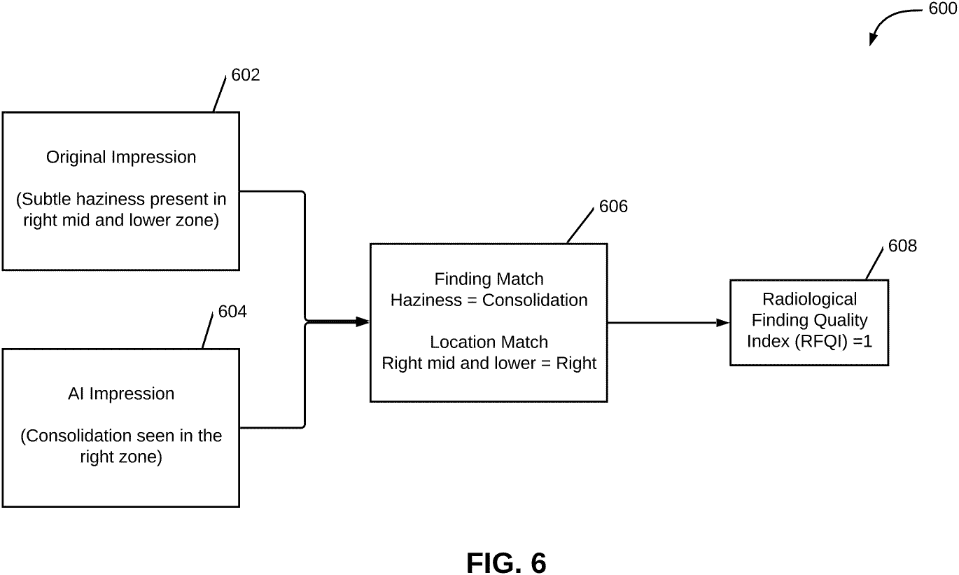

The validation module 314 may be configured to assign a match score (i.e., at step 510) to each set of the one or more sets of output tokens based on the comparison. The match score may be indicative of the degree of match between an associated set of output tokens and a corresponding set of inference tokens. The validation module 314 may be configured to determine a combined score for the output based on the match score assigned to each set of the one or more sets of output tokens. The validation module 314 may be configured to validate the output based on the combined score. The validated output may correspond to output data. The output data may be rendered as a report (a validated report) on a user device. The scoring operation is explained with an exemplary scenario in FIG. 6.

FIG. 6 is a process flow diagram 600 that shows an exemplary scenario for scoring operation in a system for validating a report generated by a report generating model, in accordance with an embodiment of the present disclosure. FIG. 6 is explained in conjunction with elements from FIG. 1 to FIG. 5.

In accordance with an embodiment, an original impression 602 of an image associated with a subject may have visual indicators that may highlight a region of interest specifically. The original impression 602 may correspond to ground truth data, for example, inference provided by a medical practitioner/doctor based on their study of the one or more images associated with a subject. In the example scenario, as shown in FIG. 6, the original impression is “Subtle haziness present in right mid and lower zone”. The region of interest may be indicative of a location of a body part/organ where an anomaly may be present. For example, subtle haziness (anomaly) present in the right mid and lower zone (location) of the body part/organ.

The AI impression 604 is the inference/prediction generated by the AI model 110 corresponding to the same image associated with the subject. For example, as shown in FIG. 6, the AI impression 604 is “Consolidation seen in the right zone”. As it will be noted, the AI impression 604 generated by the AI model 110 may not be exactly same as the original impression 602 provided by the medical practitioner/doctor based on the study of the images associated with the subject.

Thereafter, the original impression tokens may be compared with corresponding AI impression tokens to determine a degree of match. To this end, first, the original impression 602 may be tokenized to generate original impression tokens, such as “haziness”, “right”, “mid”, and “lower”. Similarly, the AIimpression 604 may be tokenized to generate AIimpression tokens, such as “consolidation” and “right”. Thereafter, the original impression tokens may be categorized into a “finding” category and a “location” category. For example, in this case, the token “haziness” is categorized in the “finding” category and a set of tokens “right”, “mid”, and “lower” are categorized in the “location” category. Further, the AI impression tokens may be categorized into the “finding” category and the “location” category. For example, in this case, the token “consolidation” is categorized in the “finding” category and the token “right” is categorized in the “location” category.

The validation module 314 may be configured to comparing each of the tokens or the sets of original impression tokens with a corresponding token or set of AI impression tokens, and assign a match score 606 to each set of the one or more sets of original impression tokens based on the comparison. The match score may be indicative of the degree of match between the original impression 602 and the AIimpression 604. In accordance with an embodiment, “haziness” from the original impression 602 may be equivalent to “consolidation” of the AI impression 604. In accordance with an embodiment, localization may be performed by the system 102 by identifying a location of the region of interest in the image. Based on the localization, the location match for “right mid and lower zone” from the original impression 602 may be equivalent to “right zone” of the AI impression 604. The match score may be referred as Radiological Finding Quality Index (RFQI) 608. The validation module 314 may be configured to determine a combined score for output based on the match score assigned to the original impression 602 and the AI impression 604. The validation module 314 may be configured to validate the output based on the combined score. The validated output may correspond to output data. The output data may be rendered as a report (a validated report) on a user device. In this example, as shown in FIG. 6, there is high degree of match between each of the original impression tokens or the sets of original impression tokens and corresponding AI impression tokens or the set of AI impression tokens. As such, a combined match score of “1” is assigned to the AI impression, and the AI impression is, therefore, validated.

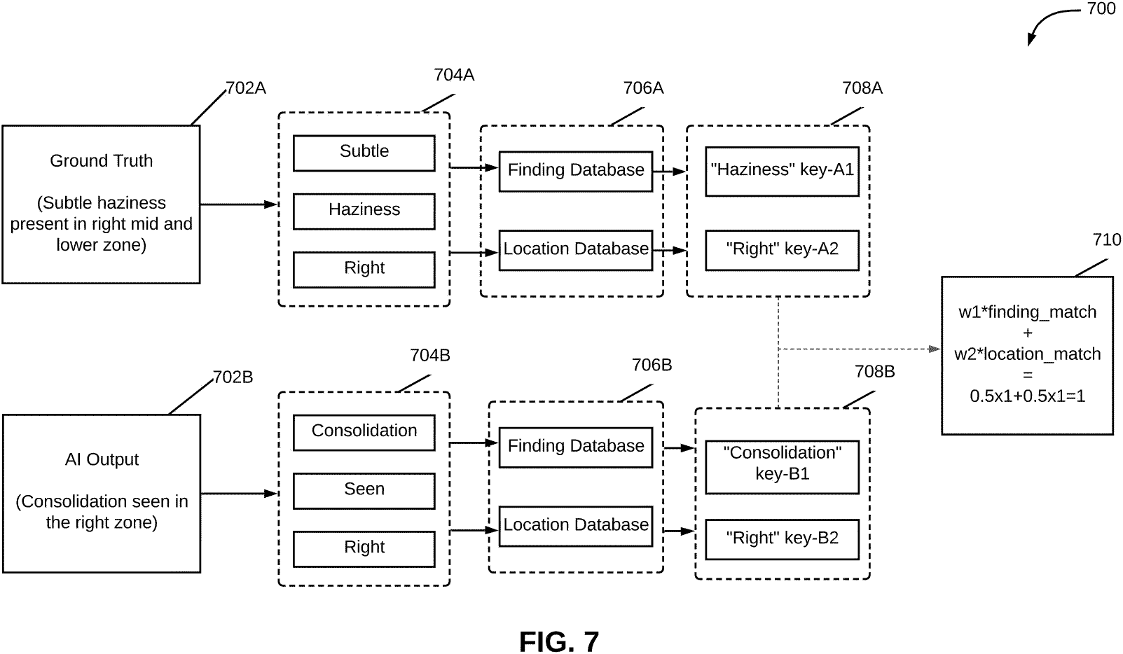

FIG. 7 is another process flow diagram 700 that shows the exemplary scenario for scoring operation for validating the report generated by AI model, in accordance with an embodiment of the present disclosure.

A ground truth 702A (corresponding to original impression 602) of an image associated with a subject is received. Further, an AI output 702B (corresponding to AI impression 604) is received. Thereafter, the ground truth 702A may be tokenized to generate ground truth tokens 704A, such as “subtle,” “haziness” and “right”. The AI output 702B may be tokenized to generate AI output tokens 704B, such as “consolidation”, “seen”, and “right”.

Further, the ground truth tokens “subtle” and “haziness” may be categorized in the “finding” category, upon referencing a database 706A (in particular, a finding database). The ground truth token “right” may be categorized in the “location” category, upon referencing the database 706A (in particular, a location database). In some embodiments, a key 708A may be selected from each of the “finding” category and the “location” category. For example, “haziness” may be selected as key-A1 and “right’ may be selected as key-A2.

Similarly, AI output tokens “consolidation” and “seen” may be categorized in the “finding” category, based on referencing with a database 706B (in particular, a finding database). The AI output token “right” may be categorized in the “location” category, based on referencing with the database 706B (in particular, a location database). Further, a key 708B may be selected from each of the “finding” category and the “location” category. For example, “consolidation” may be selected as key-B1 and “right’ may be selected as key-B2.

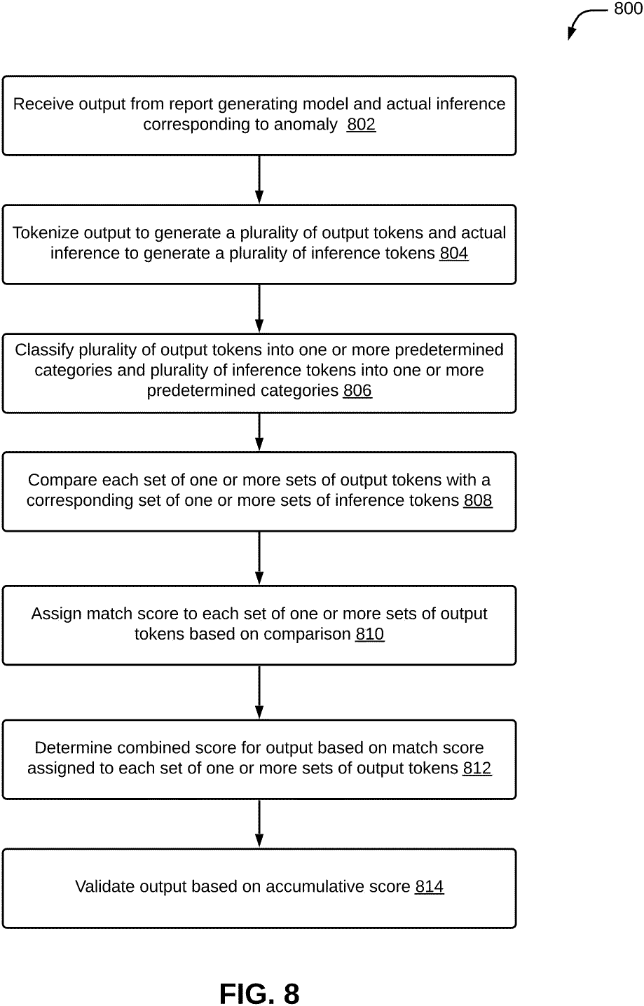

A combined match score 710 may be calculated. To this end, an individual match score may be assigned to each of the keys key-B1 and key-B2 based on their comparison with the key-A1 and key-A2, respectively, and further based on the degree of match. For example, a highest match score 0.5 may be assigned to each the keys key-B1 and key-B2 in case of high degree of match. As such, in the current example, the key-B1 and key-B2 may be assigned the highest match score 0.5 due to high degree of match. As it will be understood, the key B1 (“consolidation”) essentially matches with the key A1 (“haziness”). Similarly, the key B2 (“right”) also exactly matches with the key A2 (“right”). The combined match score 710 is calculated as a sum of the individual match scores assigned to each of the keys key-B1 and key-B2, i.e., 0.5+0.5=1, indicating a high degree of match. Therefore, the AI output is validated as being closely matching with the ground truth. FIG. 8 is a flowchart that illustrates an exemplary method of validating a report generated by a report generating model, in accordance with an embodiment of the disclosure. With reference to FIG. 8, there is shown a flowchart 800. The operations of the exemplary method may be executed by any computing system, for example, by the system 102 of FIG. 1. The operations of the flowchart 800 may start at 802 and proceed to 804.

At 802, an output from the report generating model and an actual inference corresponding to the anomaly may be received. In accordance with an embodiment, the receipt module 304 of the system 102 may be configured to receive an output from the report generating model. The output may correspond to an anomaly detected in an image. The output may comprise at least one textual sentence. In accordance with an embodiment, the receipt module 304 may be configured to receive an actual inference corresponding to the anomaly. The actual inference may comprise at least one textual sentence. The image may correspond to one of an X-ray image or a Magnetic Resonance Imaging (MRI) scan image.

At 804, the output may be tokenized to generate a plurality of output tokens and the actual inference may be tokenized to generate a plurality of inference tokens may be tokenized. The tokenization module 306 may be configured to tokenize the output to generate a plurality of output tokens. For tokenization of the output, the at least one sentence may be split into a phrase or smaller units (output tokens), such as individual words or terms. In accordance with an embodiment, the tokenization module 306 may be configured to tokenize the actual inference to generate a plurality of inference tokens.

At 806, the plurality of output tokens may be classified into one or more predetermined categories and the plurality of inference tokens may be classified into the one or more predetermined categories. In accordance with an embodiment, the classification module 310 may be configured to classify the plurality of output tokens into one or more predetermined categories to generate one or more sets of output tokens corresponding to the one or more predetermined categories. Each of the one or more sets of output tokens may comprise at least one textual element.

In accordance with an embodiment, the classification module 310 may be configured to classify the plurality of inference tokens into the one or more predetermined categories to generate one or more sets of inference tokens corresponding to the one or more predetermined categories. Each of the one or more sets of inference tokens may comprise at least one textual element.

At 808, each set of the one or more sets of output tokens may be compared with a corresponding set of the one or more sets of inference tokens. In accordance with an embodiment, the comparison module 312 may be configured to compare each set of the one or more sets of output tokens with a corresponding set of the one or more sets of inference tokens.

At 810, a match score may be assigned to each set of the one or more sets of output tokens based on the comparison. In accordance with an embodiment, the validation module 314 may be configured to assign each set of the one or more sets of output tokens based on the comparison. The match score may be indicative of the degree of match between an associated set of output tokens and a corresponding set of inference tokens.

At 812, a combined score may be determined for the output based on the match score assigned to each set of the one or more sets of output tokens. In accordance with an embodiment, the validation module 314 may be configured to determine the combined score for the output based on the match score assigned to each set of the one or more sets of output tokens.

At 814, the output may be validated based on the combined score. In accordance with an embodiment, the validation module 314 may be configured to validate the output based on the combined score. In accordance with an embodiment, the output data 320 may be rendered as a report (a validated report) on a user device. The control passes to the end.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It will be appreciated that, for clarity purposes, the above description has described embodiments of the disclosure with reference to different functional units and processors. However, it will be apparent that any suitable distribution of functionality between different functional units, processors or domains may be used without detracting from the disclosure. For example, functionality illustrated to be performed by separate processors or controllers may be performed by the same processor or controller. Hence, references to specific functional units are only to be seen as references to suitable means for providing the described functionality, rather than indicative of a strict logical or physical structure or organization.

Although the present disclosure has been described in connection with some embodiments, it is not intended to be limited to the specific form set forth herein. Rather, the scope of the present disclosure is limited only by the claims. Additionally, although a feature may appear to be described in connection with particular embodiments, one skilled in the art would recognize that various features of the described embodiments may be combined in accordance with the disclosure.

Furthermore, although individually listed, a plurality of means, elements or process steps may be implemented by, for example, a single unit or processor. Additionally, although individual features may be included in different claims, these may possibly be advantageously combined, and the inclusion in different claims does not imply that a combination of features is not feasible and/or advantageous. Also, the inclusion of a feature in one category of claims does not imply a limitation to this category, but rather the feature may be equally applicable to other claim categories, as appropriate.

What is claimed is:

1. A method of validating a report generated by a report generating model, the method comprising:
receiving:
an output from the report generating model, wherein the output corresponds to an anomaly detected in an image, and wherein the output comprises at least one textual sentence; and
an actual inference corresponding to the anomaly, wherein the actual inference comprises at least one textual sentence;
tokenizing:
the output to generate a plurality of output tokens; and
the actual inference to generate a plurality of inference tokens;
classifying:
the plurality of output tokens into one or more predetermined categories to generate one or more sets of output tokens corresponding to the one or more predetermined categories, wherein each of the one or more sets of output tokens comprises at least one textual element; and
the plurality of inference tokens into the one or more predetermined categories to generate one or more sets of inference tokens corresponding to the one or more predetermined categories, wherein each of the one or more sets of inference tokens comprises at least one textual element; and
comparing each set of the one or more sets of output tokens with a corresponding set of the one or more sets of inference tokens;
assigning a match score to each set of the one or more sets of output tokens based on the comparison, wherein the match score is indicative of the degree of match between an associated set of output tokens and a corresponding set of inference tokens;
determining a combined score for the output based on the match score assigned to each set of the one or more sets of output tokens; and
validating the output based on the combined score.

2. The method as claimed in claim 1, wherein the image is one of an X-ray image or a Magnetic Resonance Imaging (MRI) scan image.

3. The method as claimed in claim 1, further comprising:
extracting one or more features indicative of one or more anomalies from the image using a first AI model, wherein the first AI model is a Convolutional Neural Network (CNN); and
assigning a label to each of the one or more features.

4. The method as claimed in claim 3, further comprising:
generating at least one textual sentence based on the label assigned to each of the one or more features using a second AI model, whereon the second AI model is a Long Short-Term Memory (LSTM) model.

5. The method as claimed in claim 1, further comprising preprocessing:
the plurality of output tokens to extract relevant tokens from each of the one or more sets of output tokens; and
the plurality of inference tokens to extract relevant tokens from each of the one or more sets of inference tokens.

6. The method as claimed in claim 5, wherein the preprocessing of a set of output tokens or a set of inference tokens associated with a category is performed based on a historical database corresponding to the category.

7. The method as claimed in claim 5, wherein comparing each set of the one or more sets of output tokens with a corresponding set of the one or more sets of inference tokens comprises:
comparing the relevant tokens from each of the one or more sets of output tokens with the relevant tokens from a corresponding set of the one or more sets of inference tokens.

8. A system for validating a report generated by a report generating model, the system comprising:
a processor; and
a memory communicatively coupled to the processor, wherein the memory stores processor-executable instructions, which, on execution, causes the processor to:
receive:
an output from the report generating model, wherein the output corresponds to an anomaly detected in an image, and wherein the output comprises at least one textual sentence; and
an actual inference corresponding to the anomaly, wherein the actual inference comprises at least one textual sentence;
tokenize:
the output to generate a plurality of output tokens; and
the actual inference to generate a plurality of inference tokens;
classify:
the plurality of output tokens into one or more predetermined categories to generate one or more sets of output tokens corresponding to the one or more predetermined categories, wherein each of the one or more sets of output tokens comprises at least one textual element; and
the plurality of inference tokens into the one or more predetermined categories to generate one or more sets of inference tokens corresponding to the one or more predetermined categories, wherein each of the one or more sets of inference tokens comprises at least one textual element; and
compare each set of the one or more sets of output tokens with a corresponding set of the one or more sets of inference tokens;
assign a match score to each set of the one or more sets of output tokens based on the comparison, wherein the match score is indicative of the degree of match between an associated set of output tokens and a corresponding set of inference tokens;
determine a combined score for the output based on the match score assigned to each set of the one or more sets of output tokens; and
validate the output based on the combined score.

9. The system as claimed in claim 8, wherein the image is one of an X-ray image or a Magnetic Resonance Imaging (MRI) scan image.

10. The system as claimed in claim 8, wherein the the processor-executable instructions further cause the processor to:
extract one or more features indicative of one or more anomalies from the image using a first AI model, wherein the first AI model is a Convolutional Neural Network (CNN); and
assign a label to each of the one or more features.

11. The system as claimed in claim 10, wherein the the processor-executable instructions further cause the processor to:
generate at least one textual sentence based on the label assigned to each of the one or more features using a second AI model, whereon the second AI model is a Long Short-Term Memory (LSTM) model.

12. The system as claimed in claim 8, wherein the the processor-executable instructions further cause the processor to preprocess:
the plurality of output tokens to extract relevant tokens from each of the one or more sets of output tokens; and
the plurality of inference tokens to extract relevant tokens from each of the one or more sets of inference tokens.

13. The system as claimed in claim 12, wherein the preprocessing of a set of output tokens or a set of inference tokens associated with a category is performed based on a historical database corresponding to the category.

14. The system as claimed in claim 12, wherein comparing each set of the one or more sets of output tokens with a corresponding set of the one or more sets of inference tokens comprises:
comparing the relevant tokens from each of the one or more sets of output tokens with the relevant tokens from a corresponding set of the one or more sets of inference tokens.

15. A non-transitory computer-readable medium storing computer-executable instruction for generating recommendation for a user, the computer-executable instructions configured for:
receiving:
an output from the report generating model, wherein the output corresponds to an anomaly detected in an image, and wherein the output comprises at least one textual sentence; and
an actual inference corresponding to the anomaly, wherein the actual inference comprises at least one textual sentence;
tokenizing:
the output to generate a plurality of output tokens; and
the actual inference to generate a plurality of inference tokens;
classifying:
the plurality of output tokens into one or more predetermined categories to generate one or more sets of output tokens corresponding to the one or more predetermined categories, wherein each of the one or more sets of output tokens comprises at least one textual element; and
the plurality of inference tokens into the one or more predetermined categories to generate one or more sets of inference tokens corresponding to the one or more predetermined categories, wherein each of the one or more sets of inference tokens comprises at least one textual element; and
comparing each set of the one or more sets of output tokens with a corresponding set of the one or more sets of inference tokens;
assigning a match score to each set of the one or more sets of output tokens based on the comparison, wherein the match score is indicative of the degree of match between an associated set of output tokens and a corresponding set of inference tokens;
determining a combined score for the output based on the match score assigned to each set of the one or more sets of output tokens; and
validating the output based on the combined score.
